(19) United States
(12) Patent Application Publication
(10) Pub. No.: US 2004/0147619 A1
(43) Pub. Date: Jul. 29, 2004

(54) Chlorine-containing synthesis gas catalyst

(75) Inventors: Tianyan Niu, Ponca City, OK (US); Kevin L. Coy, Ponca City, OK (US); David M. Minahan, Stillwater, OK (US); Harold A. Wright, Ponca City, OK (US)

Correspondence Address: DAVID W. WESTPHAL, CONOCOPHILLIPS COMPANY, P.O. BOX 1267, PONCA CITY, OK, 74602-1267, US

(73) Assignee: ConocoPhillips Company, Houston, TX

(21) Appl. No.: 10/349,577

(22) Filed: Jan. 23, 2003

(51) Int. Cl. C01B 3/26; B01J 27/06; B01J 27/128; B01J 27/13; C10K 1/00; C07C 1/02

(52) U.S. Cl. 518/703; 423/651; 502/224; 502/230; 502/229

(57) ABSTRACT

The present invention discloses synthesis gas catalysts, and methods for making such catalysts, that are active for promoting partial oxidation of light hydrocarbons to CO and H₂. The catalysts comprise a support and an active metal. The catalysts may further comprise a promoter and halide or a rare earth oxyhalide. The present invention further discloses a method for producing synthesis gas by net partial oxidation of light hydrocarbons by contacting O₂ and light hydrocarbons in the presence of a synthesis gas catalyst as previously described. The present invention also describes a method for extending the life of a synthesis gas catalyst by contacting the catalyst with a halide. A method for making middle distillates from light hydrocarbons by partial oxidation of light hydrocarbons over a synthesis gas catalyst as previously described and Fischer-Tropsch reaction is also disclosed.

### BACKGROUND OF THE INVENTION

1. Field of the Invention

The present invention generally relates to the production of synthesis gas. More particularly, the invention relates to supported catalysts and processes for the catalytic partial oxidation of light hydrocarbons (e.g., methane or natural gas) to produce a mixture of primarily carbon monoxide and hydrogen (synthesis gas). The invention also relates to methods of preparing a catalyst or catalyst support material having properties that extend the life of synthesis gas catalysts.

2. Description of Related Art

Large quantities of methane, the main component of natural gas, are available in many areas of the world, and natural gas is predicted to outlast oil reserves by a significant margin. However, most natural gas is situated in areas that are geographically remote from population and industrial centers. The costs of compression, transportation, and storage make its use economically unattractive.

To improve the economics of natural gas use, much research has focused on methane as a starting material for the production of higher hydrocarbons and hydrocarbon liquids. The conversion of methane to hydrocarbons is typically carried out in two steps. In a first step, methane is reformed with water to produce carbon monoxide and hydrogen (i.e., synthesis gas or syngas). In a second step, the syngas intermediate is converted to higher hydrocarbon products by processes such as the Fischer-Tropsch synthesis. For example, fuels with boiling points in the middle distillate range, such as kerosene and diesel fuel, and hydrocarbon waxes may be produced from the synthesis gas.

Current industrial use of methane as a chemical feedstock proceeds by the initial conversion of methane to carbon monoxide and hydrogen by either steam reforming or dry reforming. Steam reforming currently is the major process used commercially for the conversion of methane to synthesis gas, the reaction proceeding according to Equation 1.

CH₄+H₂O←→CO+3H₂ (1)

The steam reforming reaction is endothermic (about 49 kcal/mol), requiring the expenditure of large amounts of fuel to produce the necessary heat for the industrial scale process. Another drawback of steam reforming is that for many industrial applications, the H₂:CO product ratio of 3:1 is problematic, and the typically large steam reforming plants are not practical to set up at remote sites of natural gas formations.

The catalytic partial oxidation (CPOX) of light hydrocarbons—e.g., methane or natural gas—to syngas has also been described in the literature. In catalytic partial oxidation, natural gas is mixed with air, oxygen-enriched air, or oxygen and introduced to a catalyst at elevated temperature and pressure. The partial or direct oxidation of methane yields a syngas mixture with a H₂:CO ratio of 2:1, as shown in Equation 2.

CH₄+½O₂→CO+2H₂ (2)

The H₂:CO ratio of partial oxidation is more useful than the H₂:CO ratio from steam reforming for the downstream conversion of the syngas to chemicals such as methanol or to fuels. The CPOX reaction is exothermic (−8.5 kcal/mol), in contrast to the strongly endothermic steam reforming reaction. Furthermore, oxidation reactions are typically much faster than reforming reactions. This allows the use of much smaller reactors for catalytic partial oxidation processes than is possible in a conventional steam reforming process.

While its use is currently limited as an industrial process, CPOX of methane has recently attracted much attention due to its inherent advantages, such as the fact that due to the significant heat that is released during the process, there is no requirement for the continuous input of heat in order to maintain the reaction, in contrast to steam reforming processes. An attempt to overcome some of the disadvantages and costs typical of steam reforming by production of synthesis gas via the catalytic partial oxidation of methane is described in European Patent No. 303,438. According to that method, certain high surface area monoliths coated with metals or metal oxides that are active as oxidation catalysts—e.g., Pd, Pt, Rh, Ir, Os, Ru, Ni, Cr, Co, Ce, La, and mixtures thereof—are employed as catalysts. Other suggested coating metals are noble metals and metals of groups IA, IIA, III, IV, VB, VIB, or VIIB of the periodic table of the elements.

Other methane oxidation reactions include the highly exothermic combustion (−192 kcal/mol) and partial combustion (−124 kcal/mol) reactions, Equations 3 and 4, respectively.

CH₄+2 O₂→CO₂+2H₂O (3)

CH₄+3/2 O₂→CO+2H₂O (4)

U.S. Pat. No. 5,149,464 describes a method for selectively converting methane to syngas at 650-950° C. by contacting a methane/oxygen mixture with a solid catalyst which is a d-block transition metal on a refractory support, an oxide of a d-block transition metal, or a compound of the formula MₓM′_{y}O_{z} wherein M′ is a d-block transition metal and M is Mg, B, Al, Ga, Si, Ti, Zr, Hf or a lanthanide.

U.S. Pat. No. 5,500,149 describes the combination of dry reforming and partial oxidation of methane, in the presence of added CO₂ to enhance the selectivity and degree of conversion to synthesis gas. The catalyst is a d-block transition metal or oxide such as a group VIII metal on a metal oxide support such as alumina and is made by precipitating the metal oxides, or precursors thereof such as carbonates or nitrates or any thermally decomposable salts, onto a refractory solid which may itself be massive or particulate. Alternatively, one metal oxide or precursor may be precipitated onto the other. Preferred catalyst precursors are those having the catalytic metal highly dispersed on an inert metal oxide support and in a form readily reducible to the elemental state.

For successful commercial scale operation a catalytic partial oxidation process must be able to achieve and sustain a high conversion of the methane feedstock at high gas hourly space velocities, with high selectivity for the desired H₂ and CO products. Moreover, such high conversion and selectivity levels must be achieved without detrimental effects to the catalyst, such as the formation of carbon deposits (coke) on the catalyst, which severely reduce catalyst performance. The choice of catalyst composition and the manner in which the catalyst is made are important factors in determining whether a catalyst will have sufficient physical and chemical stability to operate satisfactorily for extended periods of time on stream at moderate to high temperatures and will avoid high pressure drop in a syngas production operation.

In most of the existing syngas production processes it is difficult to select a catalyst that will be economical for large scale industrial use yet will provide the desired level of activity and selectivity for CO and H₂ and demonstrate long on-stream life. Today, metal oxide supported noble metal catalysts or mixed metal oxide catalysts are most commonly used for the selective oxidation of hydrocarbons and for catalytic combustion processes. Various techniques are employed to prepare the catalysts, including impregnation, washcoating, xerogel, aerogel or sol-gel formation, spray drying, and spray roasting. Monolith supported catalysts having pores or longitudinal channels or passageways are commonly used. Such catalyst forming techniques and configurations are well described in the literature, for example, in Structured Catalysts and Reactors, A. Cybulski and J. A. Moulijn (Eds.), Marcel Dekker, Inc., 1998, p. 599-615 (Ch. 21, X. Xu and J. A. Moulijn, “Transformation of a Structured Carrier into Structured Catalyst”).

U.S. Pat. No. 5,510,056 discloses a ceramic foam supported Ru, Rh, Pd, Os, Ir, or Pt catalyst having a specified tortuosity and number of interstitial pores that is said to allow operation at high gas space velocity. The catalyst is prepared by depositing the metal on a carrier using an impregnation technique, which typically comprises contacting the carrier material with a solution of a compound of the catalytically active metal, followed by drying and calcining the resulting material. The catalyst is employed for the catalytic partial oxidation of a hydrocarbon feedstock.

U.S. Pat. No. 5,648,582 discloses a rhodium or platinum catalyst prepared by washcoating an alumina foam monolith having an open, cellular, sponge-like structure. The catalyst is used for the catalytic partial oxidation of methane at space velocities of 120,000 h.⁻¹ to 12,000,000 h.⁻¹

More recently, particulate syngas catalysts have been found to offer certain advantages over monolithic catalysts. For example, Hohn and Schmidt (Applied Catalysis A: General 211:53-68 (2001)) compare monolith and particulate (i.e., sphere) beds in a catalytic partial oxidation process and show that a non-porous alumina support gave superior results for the production of synthesis gas, even at space velocities of 1.8×10⁶ h⁻¹, compared to a comparable alumina monolith support. The internal surface area of the support material is apparently unimportant, as the pre-sintered and unsintered alumina spheres (surface area about 10 m²/g and about 200 m²/g, respectively) gave the same results after loading with the same amount of Rh. A major advantage of those particle beds is said to be better heat transfer than the corresponding monolithic catalyst.

U.S. Patent Application No. 2001/0027258 A1 describes a catalytic partial oxidation process that includes contacting a C₁-C₄ hydrocarbon and oxygen with a particulate bed of supported Group VIII metal catalyst. The support has a surface-to-volume ratio of about 15-230 cm¹, and the preferred catalyst particle size range is about 200-2000 microns in diameter. The preferred support particles generally have a low total surface area—e.g., <20 m²/gm—and microporosity is not important to the process.

PCT Publication No. WO O₂/20395 (Conoco Inc.) describes certain rhodium-based catalysts that are active for catalyzing the net partial oxidation of methane to CO and H₂. A preferred catalyst comprises highly dispersed, high surface area rhodium on a granular zirconia support with an intermediate coating of a lanthamide and/or lanthamide oxide. The catalyst is thermally conditioned during its preparation.

Although significant advances have been made in the development of catalysts and processes for producing synthesis gas, in order for catalytic partial oxidation processes to be commercially feasible there continues to be a need for more efficient and economical processes and catalysts. At the present time, there is no commercially practical CPOX reaction system for the manufacture of syngas. The syngas production process needs to be easier to practice, not dependent upon additional ignition sources, and capable of lighting off at low temperatures. Ideal syngas catalysts would also be physically and chemically stable on stream, resist coking, and also retain a high level of conversion activity and selectivity to carbon monoxide and hydrogen under the conditions of high gas space velocity and elevated pressure that are needed for achieving high space time syngas yield.

### SUMMARY OF THE INVENTION

In accordance with certain embodiments of the present invention, supported catalysts are provided that comprise a halide. In an alternate embodiment, supported catalysts are provided that comprise a rare earth halid, more particularly a rare earth oxyhalide. In accordance with other embodiments of the present invention, methods for producing synthesis gas catalysts are provided. The methods include contacting a synthesis gas catalyst support with a metal halide, adding a promoter, and drying and calcining in such a way that a halide is left on the catalyst. Further, a method for maintaining synthesis gas catalyst activity is provided. The method includes contacting the synthesis gas catalyst with a halide. In yet another alternate embodiment, methods for producing synthesis gas from light hydrocarbons are provided. The methods include contacting a synthesis gas catalyst with a halide or using a catalyst comprising a rare earth oxyhalide. In still another embodiment of the present invention, a method is provided for converting light hydrocarbons to middle distillates or alcohols. The method includes contacting the light hydrocarbons with a synthesis gas catalyst in the presence of O₂ and a catalyst, the catalyst comprising a rare earth halide. The method further includes reacting the synthesis gas in a synthesis reaction to produce hydrocarbons and/or chemicals, for example via Fischer-Tropsch or alcohol synthesis.

### BRIEF DESCRIPTION OF THE DRAWINGS

FIG. 1 is a graph showing operating data for a synthesis gas catalyst according to the present invention.

FIG. 2 is a graph showing operating data for a similar synthesis gas catalyst without residual chloride.

### DETAILED DESCRIPTION OF PREFERRED EMBODIMENTS

According to a preferred embodiment, the present invention involves adding a halide to a synthesis gas catalyst to maintain catalyst activity. Preferably a source of chlorine atoms or ions is added to a catalyst comprising a support and an active metal. More preferably, the catalyst also contains a promoter. The active metal and the support are selected to catalyze the partial oxidation of light hydrocarbons (i.e., C₁-C₅ hydrocarbons) to produce synthesis gas (i.e., predominantly CO and H₂). Partial oxidation occurs according to Equation (2) above. It is believed that the addition of the halide maintains catalyst activity by reacting with the active metal and allowing redistribution of the active metal on the catalyst resulting in reduced deactivation due to sintering and agglomeration.

Partial Oxidation Catalysts

To accomplish the partial oxidation reaction, a light hydrocarbon feedstock and an O₂-containing gas are contacted with a catalyst that is active for catalyzing the conversion of methane or natural gas and molecular oxygen to primarily CO and H₂ by a net catalytic partial oxidation (CPOX) reaction. Preferably a very fast contact (i.e., milliseconds range)/fast quench (i.e., less than one second) reactor assembly is employed. Several schemes for carrying out catalytic partial oxidation (CPOX) of hydrocarbons in a short contact time reactor are well known and have been described in the literature. The light hydrocarbon feedstock may be any gaseous hydrocarbon having a low boiling point, such as methane, natural gas, associated gas, or other sources of predominantly C₁-C₅ hydrocarbons. The light hydrocarbon feedstock may be a gas arising from naturally occurring reserves of methane which contain carbon dioxide. Preferably, the gaseous light hydrocarbon feed comprises at least 50% by volume methane, more preferably at least 80% by volume, and still more preferably at least 90% by volume methane. The gaseous light hydrocarbon feedstock is contacted with the catalyst as a mixture with an O₂-containing gas, preferably substantially pure oxygen. The O₂-containing gas may be air, or it may also comprise steam, air, and/or CO₂ in addition to oxygen. For the purposes of this disclosure, the term “catalytic partial oxidation” or “net catalytic partial oxidation” means that the CPOX reaction (Equation (2)) predominates. However, other reactions such as steam reforming (Equation (1)), dry reforming (Equation (5)) and/or water-gas shift (Equation (6)) may also occur to a lesser extent.

CH₄+CO₂←→2CO+2H₂ (5)

CO+H₂O←→CO₂+H₂ (6)

For catalytic partial oxidation of light hydrocarbons, preferably the feed carbon to O₂ molar ratio is between 1.5:1 and 3.3:1. More preferably the ratio is between 1.7:1 and 2.1:1, and still more preferably the ratio is about 2:1.

Catalysts for carrying out catalytic partial oxidation may include an active metal, such as any group VIII metal, Re, or Zr, on a refractory support. Preferred active metals include Fe, Co, Ni, Re, Zr, Ru, Rh, Pd, Os, Ir, Pt, and combinations thereof. More preferably, the catalyst also contains a promoter, such as one or more rare earth elements (i.e., La, Ce, Pr, Nd, Pm, Sm, Eu, Gd, Th, Dy, Ho, Er, Tm, Yb, Lu, Sc, Y, or Th) or base metal promoters (e.g., Mg, Ca, Ba, Sr, Li, Na, or K).

A Sm promoter is highly preferred, followed by the other lanthamides (i.e., La, Ce, Pr, Nd, Pm, Eu, Gd, Th, Py, Ho, Er, Tm, and Yb), especially La, Yb, or Pr. Suitable support materials include refractory materials such as zirconia, magnesium stabilized zirconia, zirconia stabilized alumina, yttrium stabilized zirconia, calcium stabilized zirconia, alumina, magnesium stabilized alumina, cordierite, titania, silica, magnesia, niobia, ceria, vanadia, and silicon carbide. Preferably, the support material is alumina or zirconia.

The reaction is preferably carried out at catalyst temperatures of from about 600° C. to about 2,000° C., more preferably up to about 1,600° C. The hydrocarbon feedstock and the oxygen-containing gas are preferably preheated at a temperature between about 30° C. and 750° C., more preferably not exceeding 500° C., before contact with the catalyst to facilitate light-off of the reaction.

The process is operated at atmospheric or superatmospheric pressures, the latter being preferred. The pressures may be from about 100 kPa to about 32,000 kPa (about 1-320 atm), preferably from about 200 kPa to 10,000 kPa (about 2-100 atm). The hydrocarbon feedstock and the oxygen-containing gas may be passed over the catalyst at any of a variety of space velocities. Space velocities for the process, stated as gas hourly space velocity (GHSV), are from about 20,000 to about 100,000,000 h⁻¹, preferably from about 100,000 to about 25,000,000 h⁻¹. Although for ease in comparison with prior art systems space velocities at standard conditions have been used to describe the present invention, it is well recognized in the art that residence time is the inverse of space velocity and that the disclosure of high space velocities equates to low residence times on the catalyst. Under these operating conditions a flow rate of reactant gases is maintained sufficient to ensure a residence time of no more than 200 milliseconds with respect to each portion of reactant gas in contact with the catalyst. Preferably the residence time is less than 50 milliseconds, and more preferably under 20 milliseconds. A contact time of 10 milliseconds or less is highly preferred. The product gas mixture emerging from the reactor is harvested and may be routed directly into any of a variety of applications.

The reaction is generally carried out in a reaction vessel. The vessel may be any shape, but preferably the vessel is cylindrical. Also, the vessel may be oriented in any direction. Such a vessel may be used for fluidized bed, ebulliated bed, or fixed bed reaction conditions. Sizing of the vessel is determined by desired feed flow rate, and the technique is well known to one having ordinary skill in the art. The amount of catalyst is determined by vessel diameter and desired residence time or space velocity as is well known in the art. Also, preferably the vessel has a liner, such as a refractory liner, to protect the material of construction from the severe reaction conditions. The liner may be a ceramic material or any other material that is thermally stable at temperatures above 1,000° C.

Addition of a Halide to Partial Oxidation Catalysts to Prolong Catalyst Life

The present invention results from the unexpected discovery that adding a halide to a partial oxidation catalyst maintains catalyst activity. The halide may be added to any of the catalysts described in the previous section. The catalyst may also contain a promoter, such as a rare earth or base metal promoter, as previously described. Addition of the halide maintains catalyst activity in the CPOX reaction by a mechanism that is not fully understood. Without wishing to be bound to a particular theory, the Applicants believe that a halide-containing compound becomes mobile at reaction conditions, migrates on the catalyst surface, and deposits on the catalyst surface at a different location. When the halide-containing compound is a metal halide, the migration can cause a redispersion of the metal. This mobile compound is believed to be in vapor phase under reaction conditions. Applicants' hypothesis is supported by the detection of a metal chloride compound on the catalyst surface (SmOCl) after several calcination steps above 1000° C. during preparation and after operating in a syngas reactor for several days, and this detected metal chloride compound is different that the initial metal chloride compound that was deposited on the catalyst in the form of a rhodium salt (RhCl₃) during preparation of the catalyst.

Without limiting the scope of the present invention to any particular theory, it is believed that the presence of a halide on the partial oxidation catalyst helps reduce the effects of sintering. Sintering occurs when an active metal particle reacts with other active metal particles or with a promoter to form agglomerated particles. Agglomeration results in catalyst deactivation because the active metal is less dispersed on the catalyst and is more concentrated in fewer sites. The active metals on the catalyst tend to sinter over time under the severe partial oxidation reaction conditions. It is believed that the halide may react with the active metal to create a salt or any intermediate compound that allows the active metal to move and be redispersed on the support. Thus, sintering of catalytic metals is reduced until the supply of halide is exhausted. Experimental results indicate that without any addition of halide while the catalyst is in use, the benefit of the halide is only temporary. Thus, according to this theory, once all the halide has reacted with the available metal to redisperse the metal, the metal should sinter more quickly, resulting in more rapid catalyst deactivation.

Although a number of suitable methods are available for catalyst preparation as are well known in the art, preferably the catalyst is prepared by metal deposition and calcination. Deposition of active metal and/or promoter on the support may be accomplished by different techniques. For example, deposition methods can be impregnation, co-precipitation, chemical vapor deposition, and the like. The preferred technique for deposition is impregnation, which is typically followed by a drying step. Halide may be added in a regeneration step or while the catalyst is in service by injecting the halide in the feed, but preferably a support material is impregnated with an active metal precursor. The active metal precursor may be an active metal salt, such as an active metal halide. As previously discussed, the active metal is preferably a group VIII metal, rhenium, or zirconium, and more preferably rhodium. The support material may be any refractory support. Preferably, the support material is alumina or zirconia, which can be unmodified, modified, partially-stabilized, or stabilized. The support material is immersed in, or contacted with, a solution containing the active metal precursor, preferably an active metal halide and more preferably a rhodium halide, such as rhodium chloride. Preferably, the solution also contains a promoter precursor, such as a rare earth-containing compound, more preferably a rare earth salt. Most preferably, the promoter precursor comprises samarium. The solvent is then removed by drying, leaving behind the promoter and active metal precursors. Drying is performed at temperatures between 50 and 150° C. and pressures between about 0.05 and 10 atm, preferably between 0.05 and 2 atm for at least 0.5 hour and as much as 24 hours. Next, the support material is calcined in air at temperatures above 500° C. and pressures between about 1 and 5 atm for between 0.5 and 48 hours. Temperatures between 600 and 1100° C. and atmospheric pressure are preferred.

During calcination the active metal precursor is generally converted to an oxide, and the halide is typically removed. The present invention, however, limits the calcination conditions such that some of the halide remains. The halide removal is linked to the rate of oxygen transfer and/or calcination operating conditions. To achieve incomplete removal of the halide, one or any combination of the following steps may be used during calcination: (1) generate a high oxygen transfer resistance, such as using an oxygen feed of either low flow rate and/or low concentration; (2) reduce the calcination temperature and/or the calcination time in order to slow the rate of halide removal. During calcination the halide reacts with the promoter to form a promoter halide, preferably a rare earth halide, and more preferably samarium oxyhalide. Where rhodium chloride is used as the active metal halide precursor with samarium as a promoter, samarium oxychloride is formed on the support material.

The recitation of deposition of an active metal, a promoter, or a halide on a catalyst is not intended to mean that the particular component recited is necessarily deposited independently of other components. For example, when deposition of an active metal or an active metal precursor is recited, the active metal or active metal precursor may include a promoter, a promoter precursor, a halide, and/or other components. Thus, the recitation of deposition of a particular component is not limited to deposition of the particular component recited.

Without wishing to limit the scope of the invention, it is believed that the necessary elements for maintaining catalyst activity are the presence of a rare earth metal and a halide on the catalyst. The Applicants believe that the same benefit of maintenance of catalyst activity would be achieved whether the rare earth metal and the halide are deposited on the catalyst from a common precursor or different precursors and whether simultaneously or in different deposition steps, or using identical or different deposition techniques. The Applicants anticipate that the halide can be comprised in a promoter precursor, an active metal precursor, a non-metal halide precursor, or any combination thereof. For example, the halide and the rare earth metal can be deposited from the same rare earth halide precursor, such as samarium chloride, samarium fluoride, or lanthanum chloride. Alternatively, the halide and the rare earth metal can be deposited from two separate precursors—for example, a non-metal halide (such as hydrochloric acid or ammonium chloride) and a rare earth precursor (such as samarium nitrate hexahydrate or ammonium cerium nitrate).

Any halide may be used for the present invention. However, preferably chlorine or fluorine is used because bromine and iodine tend to be removed more quickly. More preferably, chlorine is used.

The halide may be obtained from many sources including salts or other compounds that may be used to impregnate the support. Preferably, the halide is provided with the active metal as previously described. More preferably, the active metal is rhodium and is provided for catalyst impregnation in the form RhCl₃.H₂O. This material will provide the halide and the active metal.

The halide appears to provide a benefit in catalyst life until it is entirely removed from the catalyst. Thus, no minimum quantity is required to provide a benefit. However, the halide is preferably present in a quantity greater than 1 ppm by weight, more preferably greater than 10 ppm by weight, and most preferably greater than 100 ppm by weight. Similarly, no upper limit to the effectiveness of the halide concentration is known. However, preferably no more than 100,000 ppm by weight of Cl is left on the catalyst. Downstream considerations may limit the amount of halide that may be present on the catalyst because the halide is slowly removed from the catalyst. Small amounts of halide may harm a downstream catalyst, such as a Fischer-Tropsch catalyst. Therefore, the maximum amount of halide that may be present on a catalyst depends upon downstream considerations. If a process that may be harmed is downstream of a halided catalyst, a bed of adsorbent or other halide removal material may need to be installed before that process.

The present invention is also useful for regenerating spent partial oxidation catalysts. Where metal sintering is a problem, a spent catalyst may be treated with a halide to redistribute the active metal on the support. The halide may be impregnated in the same manner as for catalyst formation, or the halide may be added to a vapor stream used for regeneration.

One such application for the CO and H₂ product stream is for producing higher molecular weight hydrocarbon compounds using Fischer-Tropsch technology. It is an advantage of the present process that efficient syngas production at superatmospheric operating pressure facilitates the direct transition to a downstream process, such as a Fischer-Tropsch process, oftentimes without the need for intermediate compression. The catalytic partial oxidation reaction may be used to convert light hydrocarbons to syngas, and the syngas may be sent to a Fischer-Tropsch process where it is converted into middle distillates such as kerosene, diesel, and/or lube oils. Alternatively, the syngas product can serve as a source of H₂ for fuel cells. Fuel cells are chemical power sources in which electrical power is generated in a chemical reaction. The most common fuel cell is based on the chemical reaction between a reducing agent such as hydrogen and an oxidizing agent such as oxygen. If providing feed for a fuel cell, a controlled-pore catalyst that provides enhanced selectivity for H₂ product may be chosen, and process variables adjusted such that a H₂:CO ratio greater than 2:1 is obtained. Also, the syngas may provide a feed for an alcohol synthesis process, such as a methanol process as is well known in the art.

Catalyst Preparation

### EXAMPLE 1

4 wt % Rh-4 wt % Sm on Al₂O₃

24.1 g samarium nitrate [Sm(NO₃)₃.6H₂O (Aldrich)] were dissolved in sufficient water to form an aqueous solution. Al₂O₃ spheres of 1 mm average diameter were immersed into the solution for wet impregnation, then allowed to dry under vacuum in a water bath held at about 70° C using a rotary evaporator (Labcono) with water recovery by condensation until water is no longer coming out of the condenser. The impregnated spheres were calcined in air according to the following schedule: 5° C./min ramp to 325° C., hold at 325° C. for 1 h, 5° C./min ramp to 700° C., hold at 700° C. for 2 h, cool down to room temperature. 19.817 g rhodium chloride [RhCl₃.xH₂O (Aldrich)] were dissolved in sufficient water to form an aqueous solution. The calcined Sm-containing spheres were immersed into the rhodium chloride solution and then dried using a rotary evaporator as described above. The dried sample was then calcined in air according to the following schedule: 5° C./min ramp to 325° C., hold at 325° C. for 1 h, 5° C./min ramp to 700° C., hold at 700° C. for 2 h, cool down to room temperature. This material was then reduced at 500° C. for 3 h under a mixed stream of 300 mL/min H₂ and 300 mL/min N₂ to provide a catalyst containing 4% Rh and 4% Sm (as determined by mass balance) supported on 1 mm alumina spheres.

### EXAMPLE 2

4 wt % Rh-4 wt % Sm on Al₂O₃

This catalyst was prepared in the same manner as Example 1, except that rhodium nitrate was used as the rhodium precursor instead of rhodium chloride (RhCl₃.xH₂O). The final composition of the catalyst is 4% Rh/4% Sm (as measured by mass balance) on the same Al₂O₃ spheres. No halide containing precursor was used in this catalyst.

Test Procedure

The partial oxidation reactions were carried out in a conventional flow apparatus. For Example 1, the apparatus used a 44 mm O.D.×38 mm I.D. quartz insert embedded inside a refractory-lined steel vessel. The quartz insert contained a catalyst bed containing Example 1 catalyst spheres held between two 80-ppi alumina foam disks. For Example 2, the apparatus used was a 19 mm O.D.×13 mm I.D. quartz insert with a 12-in length embedded inside a refractory-lined steel vessel. The quartz insert contained a catalyst bed containing Example 2 catalyst spheres held between two 45-ppi ceramic alumina foam (12 mm outside diameter×5 mm thick) disks. For both apparatuses, the upper disk acted as a radiation shield and the bottom disk acted as the catalyst bed floor to hold the spheres in place. The catalyst was tested at a natural gas to oxygen (NG:O₂) molar ratio of about 1.75-1.82 or a carbon to oxygen (C:O₂) molar ratio of about 1:85-1.92, at gas hourly space velocities (GHSV) of about 600,000-700,000 hr⁻¹, and at a pressure of about 45 psig (412 kPa). The gas hourly space velocity is defined by the volume of reactant feed at standard conditions per hour divided by the volume of catalyst. Preheating the natural gas that flowed through the catalyst bed provided the heat needed to start the reaction. Oxygen was mixed with the methane or natural gas immediately before the mixture entered the catalyst bed. Once the reaction was initiated, it proceeded autothermally. Two Type K thermocouples with ceramic sheaths were used to measure catalyst inlet and outlet temperatures. The product gas mixture was analyzed for CH₄, O₂, CO, H₂, CO₂, and N₂ using a gas chromatograph equipped with a thermal conductivity detector. A gas chromatograph equipped with a flame ionization detector analyzed the gas mixture for CH₄, C₂H₆, C₂H₄, and C₂H₂.

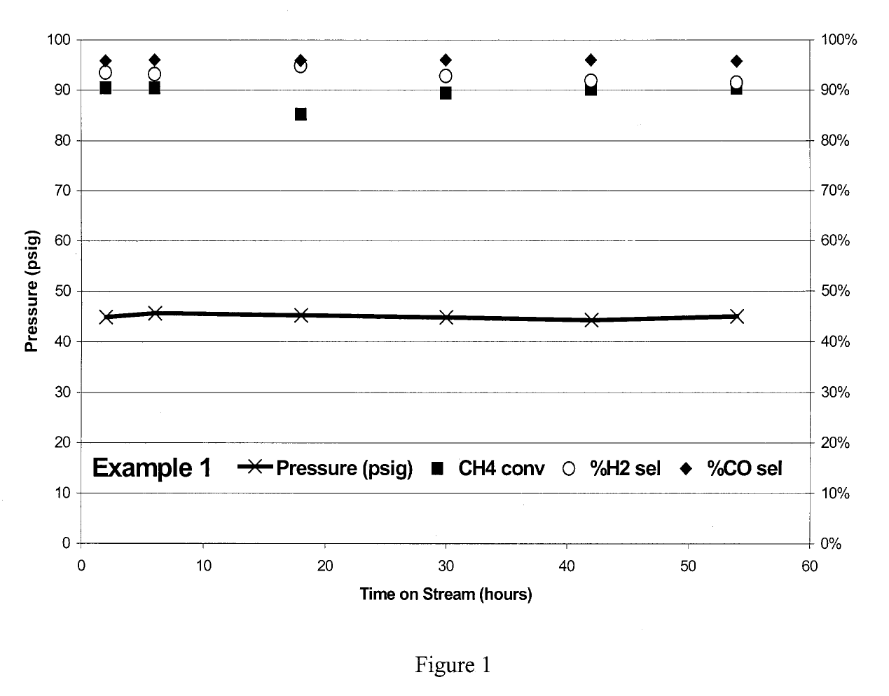

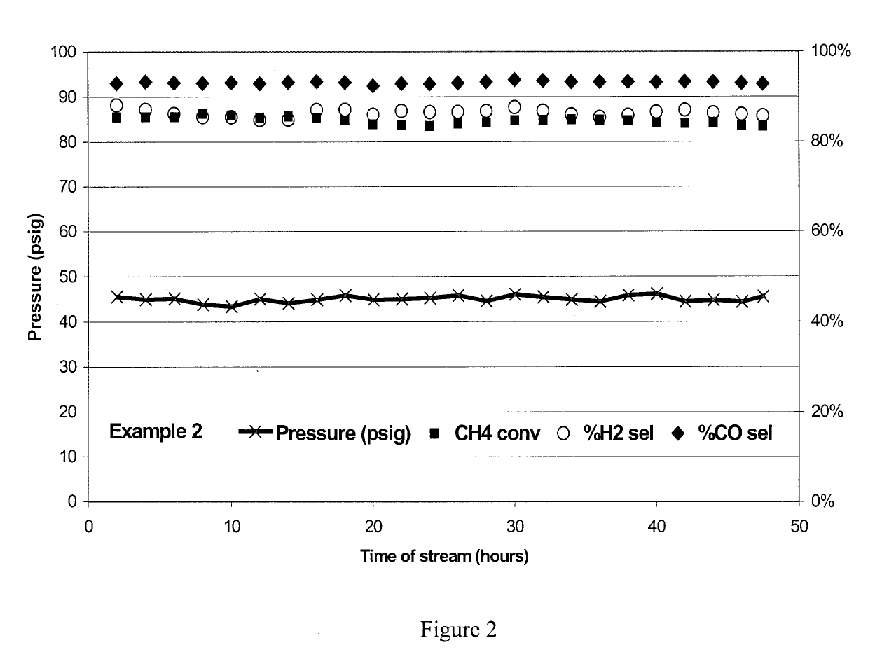

The CH₄ conversion levels and the CO and H₂ product selectivities obtained for each catalyst monolith evaluated in this test system are considered predictive of the conversion and selectivities that will be obtained when the same catalyst is employed in a commercial scale short contact time reactor under similar conditions of reactant concentrations, temperature, reactant gas pressure, and space velocity. The results of the tests are shown in Table 1 below. Further, FIG. 1 shows the operating results over time for Example 1, and FIG. 2 shows the operating results over time for Example 2. The catalyst of Example 1, having chloride remaining on it, performed better with less deactivation than the catalyst of Example 2 without chloride.

TABLE 1

| | | | | | Time | | | |
|---|---|---|---|---|---|---|---|---|
| | | | GHSV, | | on | CO | CO | H₂ |
| | | | hr⁻¹ at | Pressure, | stream | conv., | sel., | sel, |
| Example | NG:O2 | C:O2 | STP | psig | (hrs) | % | % | % |
| 1 | 1.75:1 | 1.85:1 | 700,000 | 45 | 1* | 90.4 | 95.8 | 93.4 |
| TN134405C | | | | | 54 | 90.3 | 95.8 | 91.5 |
| 2 | 1.82:1 | 1.92:1 | 600,000 | 45 | 1* | 85.4 | 93.0 | 88.1 |
| TN134501A | | | | | 47 | 83.4 | 92.8 | 85.7 |

*activity measured within the first hour

While the preferred embodiments of the invention have been shown and described, modifications thereof can be made by one skilled in the art without departing from the spirit and teachings of the invention. The embodiments described herein are exemplary only and are not intended to be limiting. Many variations and modifications of the invention disclosed herein are possible and are within the scope of the invention. The disclosures of all patents, patent applications, and publications cited above are hereby incorporated herein by reference. The discussion of certain references in the Description of Related Art is not an admission that they are prior art to the present invention, especially any references that may have a publication date after the priority date of this patent application. The recitation of steps in the following claims is not intended to imply that the steps must be performed sequentially and is not intended to imply that one step must be completed before another may be started. Further, the recitation of steps is not intended to imply that the steps must be performed independently of one another.

What is claimed is:

1. A method for producing synthesis gas comprising the steps of:
(a) combining light hydrocarbons with O₂ to form a feed stream;
(b) contacting said feed stream with a catalyst at reaction conditions, wherein said catalyst comprises a halide, an active metal, and a promoter on a refractory support.

2. The method according to claim 1 wherein said reaction conditions comprise about 600° C. to about 2,000° C. and about 100 kPa to about 32,000 kPa.

3. The method according to claim 2 wherein said reaction conditions further comprise a residence time of less than 200 milliseconds.

4. The method according to claim 1 wherein said active metal comprises rhodium.

5. The method according to claim 1 wherein said refractory support comprises alumina or zirconia.

6. The method according to claim 5 wherein said refractory support comprises alumina.

7. The method according to claim 1 wherein said promoter comprises a rare earth metal.

8. The method according to claim 7 wherein said rare earth promoter is samarium.

9. The method according to claim 1 wherein said halide comprises chlorine.

10. The method according to claim 1 wherein said O₂ comprises substantially pure O₂.

11. The method of claim 1 wherein said catalyst comprises at least 1 ppm by weight halide.

12. The method of claim 11 wherein said catalyst comprises at least 10 ppm by weight halide.

13. The method of claim 12 wherein said catalyst comprises at least 100 ppm by weight halide.

14. The method of claim 1 wherein said catalyst has an extended life compared to a similar catalyst without the halide.

15. A method for preparing a synthesis gas catalyst comprising the steps of:
(a) contacting a support with an active metal;
(b) contacting said support with a halide-containing compound;
(c) contacting said support with a promoter precursor; and
(d) drying and calcining said support in such a way that a halide remains on said support.

16. The method according to claim 15 wherein said halide-containing compound comprises an active metal chloride.

17. The method according to claim 16 wherein said active metal chloride comprises rhodium chloride.

18. The method according to claim 15 further comprising:
(e) forming a promoter oxyhalide on said support.

19. The method according to claim 18 wherein said promoter comprises a rare earth metal.

20. The method according to claim 19 wherein said promoter oxyhalide comprises samarium oxychloride.

21. The method according to claim 15 wherein said support comprises a refractory support.

22. The method according to claim 21 wherein said refractory support comprises alumina or zirconia.

23. The method according to claim 15 wherein step (c) comprises drying and calcining in such a way that said support comprises greater than 1 ppm by weight halide.

24. The method according to claim 23 wherein step (c) comprises drying and calcining in such a way that said support comprises greater than 10 ppm by weight halide.

25. The method according to claim 24 wherein step (c) comprises drying and calcining in such a way that said support comprises greater than 100 ppm by weight halide.

26. A method for extending the life of a synthesis gas catalyst, said synthesis gas catalyst comprising a support, an active metal, and a promoter, said active metal selected to promote partial oxidation of light hydrocarbons, said method comprising the step of contacting said support with a halide.

27. The method according to claim 26 further comprising reacting said halide with said promoter to form a promoter oxyhalide.

28. The method according to claim 27 wherein said promoter comprises a rare earth metal.

29. The method according to claim 28 wherein said promoter comprises samarium.

30. The method according to claim 29 wherein said halide comprises chloride.

31. The method of claim 26 wherein said active metal comprises a group VIII metal, Re, or Zr.

32. The method of claim 31 wherein said active metal comprises rhodium.

33. The method of claim 26 wherein said support comprises a refractory support.

34. The method of claim 33 wherein said support comprises alumina or zirconia.

35. The method of claim 34 wherein said support comprises alumina.

36. A catalyst comprising:
a support;
an active metal; and
a rare earth oxyhalide.

37. The catalyst according to claim 36 wherein said active metal is selected from the group consisting of Fe, Co, Ni, Re, Zr, Ru, Rh, Pd, Os, Ir, Pt, and combinations thereof.

38. The catalyst according to claim 37 wherein said active metal is Rh.

39. The catalyst according to claim 36 wherein said support comprises a refractory support.

40. The catalyst according to claim 36 wherein said rare earth oxyhalide comprises rare earth oxychloride.

41. The catalyst according to claim 40 wherein said rare earth oxychloride comprises samarium oxychloride.

42. The catalyst according to claim 36 wherein said rare earth oxyhalide comprises a halide and wherein said catalyst comprises at least 1 ppm by weight of said halide.

43. The catalyst according to claim 42 wherein said catalyst comprises at least 10 ppm by weight of said halide.

44. The catalyst according to claim 43 wherein said catalyst comprises at least 100 ppm by weight of said halide.

45. A method for making synthesis gas comprising the steps of:
(a) forming a mixture of O₂ and light hydrocarbons; and
(b) contacting said mixture with a catalyst, said catalyst comprising
a support;
an active metal; and
a rare earth oxyhalide.

46. The method according to claim 45 wherein said active metal is selected from the group consisting of Fe, Co, Ni, Re, Zr, Ru, Rh, Pd, Os, Th, Pt, and combinations thereof.

47. The method according to claim 46 wherein said active metal is Rh.

48. The method according to claim 45 wherein said support comprises a refractory support.

49. The method according to claim 45 wherein said rare earth oxyhalide comprises rare earth oxychloride.

50. The method according to claim 49 wherein said rare earth oxychloride comprises samarium oxychloride.

51. A method of converting light hydrocarbons to middle distillates comprising the steps of:
(a) converting said light hydrocarbons to syngas by net partial oxidation reaction in the presence of a catalyst, said catalyst comprising
a support;
an active metal; and
a rare earth oxyhalide; and
(b) converting said syngas to middle distillates by Fischer-Tropsch reaction.

52. The method according to claim 51 wherein said active metal is selected from the group consisting of Fe, Co, Ni, Re, Zr, Ru, Rh, Pd, Os, Ir, Pt, and combinations thereof.

53. The method according to claim 52 wherein said active metal is Rh.

54. The method according to claim 51 wherein said support comprises a refractory support.

55. The method according to claim 51 wherein said rare earth oxyhalide comprises rare earth oxychloride.

56. The method according to claim 55 wherein said rare earth oxychloride comprises samarium oxychloride.

57. The method according to claim 51 wherein said rare earth oxyhalide comprises a halide and wherein said catalyst comprises at least 1 ppm by weight halide.

58. The method according to claim 57 wherein said catalyst comprises at least 10 ppm by weight halide.

59. The method according to claim 58 wherein said catalyst comprises at least 100 ppm by weight halide.

60. A method of converting light hydrocarbons to alcohols comprising the steps of:
(a) converting said light hydrocarbons to syngas by net partial oxidation reaction in the presence of a catalyst, said catalyst comprising
a support;
an active metal; and
a rare earth oxyhalide; and
(b) converting said syngas to alcohols.

61. The method according to claim 60 wherein said alcohols comprise methanol.

62. The method according to claim 60 wherein said active metal is selected from the group consisting of Fe, Co, Ni, Re, Zr, Ru, Rh, Pd, Os, Ir, Pt, and combinations thereof.

63. The method according to claim 62 wherein said active metal is Rh.

64. The method according to claim 60 wherein said support comprises a refractory support.

65. The method according to claim 60 wherein said rare earth oxyhalide comprises rare earth oxychloride.

66. The method according to claim 65 wherein said rare earth oxychloride comprises samarium oxychloride.

67. The method according to claim 60 wherein said rare earth oxyhalide comprises a halide and wherein said catalyst comprises at least 1 ppm by weight halide.

68. The method according to claim 67 wherein said catalyst comprises at least 10 ppm by weight halide.

69. The method according to claim 68 wherein said catalyst comprises at least 100 ppm by weight halide.

70. A method for preparing a synthesis gas catalyst comprising the steps of:
(a) depositing a rare earth metal precursor on a support;
(b) depositing a halide on said support;
(c) depositing an active metal precursor on said support;
(d) optionally, drying said support after step (a), (b), or (c); and
(e) calcining said support after step (a), (b), or (c) such that an effective amount of halide remains on said support.

71. The method according to claim 70 wherein steps (a), (b), and (c) are performed in order.

72. The method according to claim 70 wherein steps (a), (b), and (c) are performed simultaneously.

73. The method according to claim 70 wherein two steps out of steps (a), (b) and (c) are performed simultaneously.

74. The method according to claim 73 wherein steps (b) and (a) comprise depositing a rare earth metal halide on a support.

75. The method according to claim 73 wherein steps (b) and (c) comprise depositing an active metal halide on said support.

76. The method according to claim 70 wherein said active metal precursor comprises a Group VIII metal, Re, or Zr.

77. The method according to claim 71 wherein said active metal precursor comprises rhodium.

78. The method according to claim 70 wherein said rare earth metal precursor comprises samarium or lanthanum.

79. The method according to claim 70 wherein said halide is chloride or fluoride.

80. The method according to claim 70 wherein said halide comprises a non-metal halide compound.

81. The method according to claim 80 wherein said non-metal halide compound is hydrochloric acid or ammonium chloride.

82. The method according to claim 70 wherein steps (b) and (c) comprise depositing an active metal halide on said support.

83. The method according to claim 70 wherein steps (a) and (b) comprise depositing a rare earth metal halide on a support.

84. The method according to claim 70 wherein steps (a), (b), and (c) comprise a method selected from the list consisting of impregnation, co-precipitation, chemical vapor deposition, and any combination thereof.

85. The method according to claim 84 wherein steps (a), (b), and (c) comprise the method of impregnation.

86. The method according to claim 70 wherein said support comprises a refractory support.

87. The method according to claim 71 wherein said refractory support comprises alumina or zirconia.

88. The method according to claim 70 further comprising
(f) reducing said support after step (e).

89. The method according to claim 70 wherein step (b) comprises depositing a halide on said catalyst such that said catalyst comprises at least 1 ppm by weight halide.

90. The method according to claim 89 wherein step (b) comprises depositing a halide on said catalyst such that said catalyst comprises at least 10 ppm by weight halide.

91. The method according to claim 90 wherein step (b) comprises depositing a halide on said catalyst such that said catalyst comprises at least 100 ppm by weight halide.
